(19) United States
(12) Patent Application Publication
(10) Pub. No.: US 2025/0375358 A1
(43) Pub. Date: Dec. 11, 2025

(54) COMPOSITE RESIN TOOTH CONTAINING NON-CROSSLINKED POLYMER PARTICLES

(71) Applicant: SHOFU INC., Kyoto (JP)

(72) Inventors: Masaaki MORIKAWA, Kyoto (JP); Yoshiyuki JOGETSU, Kyoto (JP); Yuji SADAKANE, Kyoto (JP); Katsuya KIMOTO, Kyoto (JP)

(73) Assignee: SHOFU INC., Kyoto (JP)

(21) Appl. No.: 19/086,609

(22) Filed: Mar. 21, 2025

(30) Foreign Application Priority Data
Mar. 23, 2024 (JP) 2024-047382

(51) Int. Cl. A61K 6/887; A61K 6/60; A61K 6/70

(52) U.S. Cl. CPC A61K 6/887; A61K 6/60; A61K 6/70

(57) ABSTRACT

To provide a composite resin tooth that has excellent fracture resistance capable of withstanding the occlusal pressure in the oral cavity and exhibits good adhesive property to a denture base.

To provide a composite resin tooth having a single layer structure or a layer structure of two or more layers, comprising
a composite resin layer consisting of a polymerized and cured product of a curable composition including polymerizable monomer (A), organic-inorganic composite filler (B), inorganic fine particle (C) and non-crosslinked polymer particle (D), wherein
the content of the non-crosslinked polymer particle (D) in the curable composition is within a range of 1% by mass or more and 5% by mass or less, and
an average particle diameter of all of the inorganic particles (C) contained in the curable composition is 1 μm or less.

### CROSS-REFERENCE TO RELATED APPLICATIONS

This application is based on and claims the benefit of priority from Japanese Patent Application Serial No. 2024-047382 (filed on Mar. 23, 2024), the contents of which are hereby incorporated by reference in their entirety.

### BACKGROUND OF THE INVENTION

### Field of the Invention

The present disclosure relates to a composite resin tooth as an artificial tooth for use in manufacturing dentures.

### Description of the Related Art

Conventionally, a resin tooth manufactured by mixing methyl methacrylate and polymethyl methacrylate and then polymerizing and curing them has been used as an artificial tooth. Although a resin tooth has excellent transparency, moldability and adhesive property to a denture base, there has been problems in low surface hardness and easy wear in the oral cavity.

In contrast, a composite resin tooth has a composite resin layer manufactured by polymerizing and curing a curable composition containing a polyfunctional (meth)acrylate-based polymerizable monomer and inorganic fine particles and/or an organic-inorganic composite filler, and therefore exhibit high surface hardness and excellent abrasion resistance.

Japanese Patent No. 2517753 and Japanese Patent No. 5804517 disclose composite resin teeth in which resistance to water absorption and resistance to discoloration and coloring are improved by forming a composite resin layer from a curable composition containing a specific polymerizable monomer.

### SUMMARY OF THE INVENTION

### Technical Problem

The composite resin teeth disclosed in Japanese Patent No. 2517753 and Japanese Patent No. 5804517 have improved mechanical properties compared to resin teeth. However, in the case that the opposing tooth is a prosthesis made of ceramics, zirconia, or other material with extremely high surface hardness, fracture resistance to withstand the occlusal pressure in the oral cavity is insufficient.

In addition, the composite resin layer of these composite resin teeth has poor adhesive property to the denture base, tiny clearance form at the interface between the artificial teeth and the denture base in the case of using in the mouth. Therefore, there is a case where plaque and pigment is deposited in the clearance to adversely affect appearance and hygiene.

Therefore, an object of the present disclosure is to provide a composite resin tooth that has excellent fracture resistance capable of withstanding the occlusal pressure in the oral cavity and exhibits good adhesive property to a denture base.

### Solution to Problem

In order to achieve the above described objectives, the present disclosures made extensive study and as a result, it has been found that by compounding a specific amount of non-crosslinked polymer particles to the composite resin layer of a composite resin tooth, high fracture resistance and excellent adhesive property to the denture base can be exhibited, thereby completing the present disclosure. More specifically, in the composite resin tooth of the present disclosure, the non-crosslinked polymer particles compounded to the composite resin layer absorb the stress transmitted to the composite resin layer in the case of being subjected to occlusal pressure, thereby preventing the generation of fracture. In addition, be the presence of non-crosslinked polymer particles, it is possible to improve adhesive property to the denture base compared to the composite resin layer in conventional composite resin tooth and to suppress the generation of tiny clearance at the interface between the composite resin layer and the denture base, which are the cause of deposition of plaque, pigments and the like.

That is, the above problem can be solved by the following component composition. A composite resin tooth having a single layer structure or a layer structure of two or more layers, comprising
a composite resin layer consisting of a polymerized and cured product of a curable composition including polymerizable monomer (A), organic-inorganic composite filler (B), inorganic fine particle (C) and non-crosslinked polymer particle (D), wherein
the content of the non-crosslinked polymer particle (D) in the curable composition is within a range of 1% by mass or more and 5% by mass or less, and
an average particle diameter of all of the inorganic particles (C) contained in the curable composition is 1 μm or less.

### Advantageous Effects of Invention

The present disclosure can provide a composite resin tooth that has excellent fracture resistance capable of withstanding the occlusal pressure in the oral cavity and exhibits good adhesive property to a denture base.

### BRIEF DESCRIPTION OF DRAWINGS

FIG. 1 Schematic view showing the structure of the test specimen

FIG. 2 Diagram showing the test state of a test specimen attached to a dedicated jig

### DETAILED DESCRIPTION OF THE PREFERRED EMBODIMENTS

The present disclosure will be described in detail below. In the present specification, a composite resin tooth means an artificial tooth having at least one composite resin layer, which will be described later. In the present specification, a composite resin layer means one of the layer constituting a composite resin tooth, and means a layer formed by polymerizing and curing a curable composition containing a polymerizable monomer and an organic-inorganic composite filler and/or an inorganic fine particle.

In the present specification, the non-crosslinked polymer particle refers to a particle of a polymer of one or more types of monofunctional polymerizable monomers, and in particular, refers to a particle of a polymer having no crosslinking points between polymers.

In the present specification, the term “(meth)acrylate” inclusively refers to both acrylate and methacrylate, the term “(meth)acryloyl” inclusively refers to both acryloyl and methacryloyl, the term “(meth)acrylic acid” inclusively refers to both acrylic acid and methacrylic acid, and the term “(meth)acrylamide” inclusively refers to both acrylamide and methacrylamide.

In addition, in the present specification term “average particle diameter” means a particle diameter at which an integrated value from the small particle diameter side becomes 50% (D50)in a volume-based particle diameter distribution measured using a laser diffraction/scattering type particle size distribution measuring device.

In the composite resin tooth having a single layer structure or a layer structure of two or more layers of the present disclosure, the composite resin layer is formed by polymerizing and curing a curable composition containing polymerizable monomer (A), organic-inorganic composite filler (B), inorganic fine particle (C) and a specific amount of non-crosslinked polymer particle (D). These components will be described in detail below.

In the present disclosure, the average particle diameter of the non-crosslinked polymer particle (D) may be 5 μm or more and 50 μm or less.

In the present disclosure, the content of the inorganic fine particles (C) in the curable composition may be within a range of 15% by mass or more and 35% by mass or less, and the content of the inorganic filler (b-1) contained in the organic-inorganic composite filler (B) may be within a range of 10% by mass or more and 35% by mass or less.

In the present disclosure, the content of methyl methacrylate with respect to the whole of the polymerizable monomer (A) may be 5 mass % or less.

In the present disclosure, the non-crosslinked polymer particle (D) may include a polymethyl methacrylate particle.

As the (A) polymerizable monomer that can be used in the curable composition for forming the composite resin layer (hereinafter referred to as the “curable composition of the present disclosure”), any known polymerizable monomer can be used without particularly limitation in terms of its molecular structure. That is, specific examples of a polymerizable unsaturated group contained in the polymerizable monomer (A) include a (meth) acryloyloxy group, a (meth) acrylamide group, a styryl group, a vinyl group and an allyl group, but are not limited thereto. Among these polymerizable unsaturated groups, a (meth) acryloyloxy group and a (meth) acrylamide group are preferable because of its excellent polymerization rate. In addition, there is no particular limitation on the number of polymerizable unsaturated groups contained in the polymerizable monomer (A). The hydrocarbon group bonded to the polymerizable unsaturated group may be any of an aliphatic hydrocarbon group, an alicyclic hydrocarbon group, an aromatic hydrocarbon group and a combination thereof, and the hydrocarbon group may have any substituent such as an acidic group, a hydroxyl group, a halogen atom, a sulfur atom, an alkoxy group, an amino group and a glycidyl group. Specific examples of the polymerizable monomer (A) are as follows.

Specific examples of the monofunctional polymerizable monomer include (meth) acrylic acid esters such as (meth) acrylic acid, methyl (meth) acrylate, ethyl (meth) acrylate, isopropyl (meth) acrylate, n-propyl (meth) acrylate, isobutyl (meth) acrylate, n-butyl (meth) acrylate, t-butyl (meth) acrylate, sec-butyl (meth) acrylate, n-amyl (meth) acrylate, isoamyl (meth) acrylate, n-hexyl (meth) acrylate, isodecyl (meth) acrylate, lauryl (meth) acrylate, stearyl (meth) acrylate, 2-ethylhexyl (meth) acrylate, cyclohexyl (meth) acrylate, adamantyl (meth) acrylate, phenyl (meth) acrylate, phenoxy diethyleneglycol (meth) acrylate, methoxy polyethylene glycol (meth) acrylate, benzyl (meth) acrylate, 2-phenylethyl (meth) acrylate, o-phenoxybenzyl (meth) acrylate, m-phenoxybenzyl (meth) acrylate, p-phenoxybenzyl (meth) acrylate, tetrahydrofurfuryl (meth) acrylate, glycidyl (meth) acrylate, isobornyl (meth) acrylate, allyl (meth) acrylate, 2-methoxyethyl (meth) acrylate, 2-ethoxyethyl (meth) acrylate, phenoxyethyl (meth) acrylate, 2-hydroxyethyl (meth) acrylate, 2-hydroxypropyl (meth) acrylate, 3-hydroxypropyl (meth) acrylate, 2-hydroxybutyl (meth) acrylate, glycerol (meth) acrylate, (meth) acryloyloxyethyl methyl succinate, 2-(meth) acryloyloxyethyl propionate, acetoacetoxyethyl (meth) acrylate, acetoacetoxypropyl (meth) acrylate and acetoacetoxybutyl (meth) acrylate; silane compounds such as γ-(meth) acryloyloxypropyl trimethoxysilane and γ-(meth) acryloyloxypropyl triethoxysilane; amines such as 2-(N,N-dimethylamino) ethyl (meth) acrylate and 2-(N,N-diethylamino) ethyl (meth) acrylate; fluorine-containing (meth) acrylates such as 2,2,2-trifluoroethyl (meth) acrylate, perfluorohexylethyl (meth) acrylate and perfluorooctylethyl (meth) acrylate and (meth) acrylamides thereof, and N-methylol (meth) acrylamide.

Specific examples of the aromatic bifunctional polymerizable monomer include 2,2-bis [4-[3-(meth) acryloyloxy-2-hydroxypropoxy]phenyl]propane, 2,2-bis (4-(meth) acryloyloxyphenyl) propane, 2,2-bis (4-(meth) acryloyloxy ethoxyphenyl) propane, 2,2-bis (4-(meth) acryloyloxy diethoxyphenyl) propane, 2,2-bis (4-(meth) acryloyloxy tetraethoxyphenyl) propane, 2,2-bis (4-(meth) acryloyloxy pentaethoxyphenyl) propane, 2,2-bis (4-(meth) acryloyloxy dipropoxyphenyl) propane, 2-(4-(meth) acryloyloxy ethoxyphenyl)-2-(4-(meth) acryloyloxy diethoxyphenyl) propane, 2-(4-(meth) acryloyloxy diethoxyphenyl)-2-(4-(meth) acryloyloxy triethoxyphenyl) propane, 2-(4-(meth) acryloyloxy dipropoxyphenyl)-2-(4-(meth) acryloyloxy triethoxyphenyl) propane, 2,2-bis (4-(meth) acryloyloxy dipropoxyphenyl) propane, 2,2-bis (4-(meth) acryloyloxy isopropoxyphenyl) propane, 2,2-bis (4-(meth) acryloyloxy polyethoxyphenyl) propane, 9,9-bis [4-(2-(meth) acryloyloxy ethoxy)phenyl] fluorene, and (meth) acrylamides thereof.

Specific examples of the aliphatic bifunctional polymerizable monomer include ethylene glycol di (meth) acrylate, diethylene glycol di (meth) acrylate, triethylene glycol di (meth) acrylate, tetraethylene glycol di (meth) acrylate, polyethylene glycol di (meth) acrylate, dipropylene glycol di (meth) acrylate, tripropylene glycol di (meth) acrylate, neopentyl glycol di (meth) acrylate, 3-methyl-1,5-pentanediol di (meth) acrylate, 1,3-butanediol di (meth) acrylate, 1,4-butanediol di (meth) acrylate, 1,6-hexanediol di (meth) acrylate, 1,9-nonanediol di (meth) acrylate, 1,10-decanediol di (meth) acrylate, tricyclodecane dimethanol di (meth) acrylate, glycerol-1,3-di (meth) acrylate, 3-hydroxypropyl-1,2-di (meth) acrylate, 2-hydroxy-3-acryloyloxypropyl (meth) acrylate, 1,2-bis (3-(meth) acryloyloxy-2-hydroxypropoxy) ethane, 1,2-bis (3-(meth) acryloyloxy-2-hydroxypropoxy) propane, 2-hydroxy-1,3-bis (3-(meth) acryloyloxy-2-hydroxypropoxy) propane and (meth) acrylamides thereof.

Specific examples of the tri or more functional polymerizable monomer include trimethylolpropane tri (meth) acrylate, trimethylolethane tri (meth) acrylate, trimethylolmethane tri (meth) acrylate, pentaerythritol tri (meth) acrylate, glycerin tri (meth) acrylate, pentaerythritol tetra (meth) acrylate, dipentaerythritol tetra(meth) acrylate, dipentaerythritol penta (meth) acrylate, dipentaerythritol hexa (meth) acrylate, ditrimethylolpropane tetra (meth) acrylate and (meth) acrylamides thereof.

Specific examples of the urethane-based polymerizable monomer include (meth) acrylate compounds having a urethane linkage, which are derived from an adduct of a polymerizable monomer having a hydroxyl group such as 2-hydroxyethyl (meth) acrylate, 2-hydroxypropyl (meth) acrylate and 3-chloro-2-hydroxypropyl (meth) acrylate, and an isocyanate compound such as methylcyclohexane diisocyanate, methylene bis (4-cyclohexyl isocyanate), hexamethylene diisocyanate, trimethylhexamethylene diisocyanate, isophorone diisocyanate, diisocyanate methylmethylbenzene and 4,4-diphenylmethane diisocyanate.

Furthermore, in addition to the above described polymerizable monomers, oligomers or polymers having at least one polymerizable group may be used. The polymerizable monomer (A) is not limited the above described and may be used alone or in combination of plurality thereof.

There are no particular limitations on the content of the polymerizable monomer (A), but is preferably within the range of 10% by mass or more and 50% by mass or less, more preferably within the range of 25% by mass or more and 50% by mass or less, and further preferably within the range of 25% by mass or more and 40% by mass or less, in the curable composition of the present disclosure. When the content of the polymerizable monomer (A) is less than 10% by mass, there is a case where the fracture resistance of the composite resin layer decreases. When the content of the polymerizable monomer (A) is more than 50% by mass, there is a case where the surface hardness, the abrasion resistance, the compressive strength and the like decrease.

When the polymerizable monomer (A) contains methyl methacrylate, it is preferable that the content of methyl methacrylate is 5 mass % or less with respect to the whole of the polymerizable monomer (A). When the content of methyl methacrylate exceeds 5 mass % there is a case where the surface hardness, the abrasion resistance, the compressive strength, the fracture resistance and the like of the composite resin layer decrease. It is preferable that the curable compositions of the present disclosure does not contain methyl methacrylate.

The (B) organic-inorganic composite filler that can be used in the curable composition of the present disclosure is a composite particle consisting of an inorganic portion contained in the form of an inorganic filler (b-1) and an organic portion where a polymerizable monomer (b-2) is cured, and the inorganic filler (b-1) exists in a dispersed state in the cured polymerizable monomer (b-2). The organic-inorganic composite filler (B) can be obtained by making an inorganic filler (b-1) and a polymerizable monomer (b-2) containing a polymerization initiator in as homogeneous a state as possible, curing the polymerizable monomer (b-2), and pulverizing the cured product as necessary.

An inorganic filler (b-1) that can be used in the manufacture of the (B) organic-inorganic composite filler will be described. The inorganic filler (b-1) is not particularly limited in terms of its constituent elements, and any known inorganic filler can be used. Specific examples of the inorganic filler (b-1) include inorganic oxides such as silica, alumina, titania, zirconia, strontium oxide, barium oxide, yttrium oxide, lanthanum oxide and ytterbium oxide; inorganic complex oxides such as silica-zirconia, silica-titania, silica-titania-barium oxide and silica-titania-zirconia; glasses such as molten silica, quartz, aluminosilicate glass, fluoroaluminosilicate glass, borosilicate glass, alminoborate glass and boroaluminosilicate glass; and metallic fluorides such as calcium fluoride, barium fluoride, strontium fluoride, yttrium fluoride, lanthanum fluoride and ytterbium fluoride.

A shape of these of the inorganic filler (b-1) is not particularly limited, and may be any shape such as spherical, needle-like, plate-like, ground-like and scaly-shapes, and aggregate thereof may be used without any problems. The above described inorganic filler (b-1) is not limited to these and may be used alone or in a combination of plurality thereof.

There are no particular limitations on the particle diameter of the inorganic filler (b-1), but in consideration of the balance of various properties in the composite resin layer, it is preferable that the average particle diameter is 0.005 μm or more and 3 μm or less. When the average particle diameter of the inorganic filler (b-1) is less than 0.005 μm, there is a case where the inorganic filler (b-1) aggregates remarkably and it makes difficult to uniformly disperse the inorganic filler (b-1) in the organic-inorganic composite filler (B), and therefore the compressive strength and fracture resistance of the composite resin layer decrease. Furthermore, when the average particle diameter of the inorganic filler (b-1) exceeds 3 μm, there is a case where the polishing property of the composite resin layer decreases, a smooth surface is not obtained and coloring easily occurs. In the curable composition of the present disclosure, the inorganic filler (b-1) constituting the organic-inorganic composite filler (B) may consist of only an inorganic filler having an average particle diameter of 0.005 μm or more and 3 μm or less.

It is preferable that these inorganic fillers (b-1) are subjected to a surface treatment to be made hydrophobic. This surface treatment enables high filling of the inorganic filler (b-1) in the organic-inorganic composite filler (B) to improve the mechanical characteristic of the organic-inorganic composite filler (B) itself. The surface treatment agent that can be used for the surface treatment of the inorganic filler (b-1) is not particularly limited, and known agents such as an organosilicon compound, an organozirconium compound, an organotitanium compound and organoaluminum compound can be used, but the most commonly used is an organosilicon compound. Specific examples of the organosilicon compound include methyltrimethoxysilane, ethyltrimethoxysilane, methoxytripropylsilane, propyltriethoxysilane, hexyltrimethoxysilane, vinyltrimethoxysilane, vinyltriethoxysilane, vinyltrichlorosilane, vinyltri (β-methoxyethoxy) silane, γ-(meth) acryloyloxypropyl trimethoxysilane, 8-(meth) acryloyloxyoctyl trimethoxysilane, γ-glycidoxypropyl trimethoxysilane, γ-mercaptopropyl trimethoxysilane, γ-aminopropyl trimethoxysilane, 3-aminopropyl triethoxysilane, methyltrichlorosilane, phenyltrichlorosilane, trimethylsilylisocyanate, vinylsilyltriisocyanate, phenylsilyltriisocyanate and hexamethyldisilazane, but are not limited thereto. These surface treatment agents can be used alone or in a combination of a plurality thereof. The method of surface treatment is not particularly limited, and any known method can be applied. Furthermore, the amount of the surface treatment agent relative to the inorganic filler (b-1) when performing the surface treatment is not particularly limited, and may be appropriately adjusted depending on the particle diameter of the inorganic filler (b-1) and the like.

It is preferable that a content of the inorganic filler (b-1) contained in the raw material of the organic-inorganic composite filler (B) is preferably 8% by mass or more and 50% by mass or less and more preferably 10% by mass or more and 35% by mass or less. When the content of the inorganic filler (b-1) in the organic-inorganic composite filler (B) is less than 8% by mass, there is a case where the surface hardness, the abrasion resistance and the compressive strength and the like of the composite resin layer decrease. When the content of the inorganic filler (b-1) is more than 50% by mass, there is a case where the brittleness of the organic-inorganic composite filler (B) increases and the fracture resistance of the composite resin layer decreases. In the curable composition of the present disclosure, the organic-inorganic composite filler (B) may contain 8% by mass or more and 50% by mass or less of an inorganic filler (b-1) having an average particle diameter of 0.005 μm or more and 3 μm or less. The curable composition of the present disclosure may contain only an organic-inorganic composite filler in which the content of the inorganic filler (b-1) contained in the raw material is 8% by mass or more and 50% by mass or less, as the organic-inorganic composite filler (B). The curable composition of the present disclosure may contain only an organic-inorganic composite filler containing 8% by mass or more and 50% by mass or less of the inorganic filler (b-1) having an average particle diameter of 0.005 μm or more and 3 μm or less, as the organic-inorganic composite filler (B).

The molecular structure of the polymerizable monomer (b-2) that can be used to manufacture the organic-inorganic composite filler (B) is not particularly limited, and the same polymerizable monomer as the above described (A) polymerizable monomer can be used.

It is preferable that a content of the polymerizable monomer (b-2) contained in the raw material of the organic-inorganic composite filler (B) is 48% by mass or more and 90% by mass or less and more preferably 63% by mass or more and 88% by mass or less. When the content of the polymerizable monomer (b-2) in the organic-inorganic composite filler (B) is less than 48% by mass, there is a case where the brittleness of the organic-inorganic composite filler (B) increases and the fracture resistance of the composite resin layer decreases. On the other hand, when the content of the polymerizable monomer (b-2) exceeds 90% by mass, there is a case where the surface hardness, abrasion resistance, compressive strength and the like of the composite resin layer decrease.

Next, a polymerization initiator that can be used in the manufacture of the organic-inorganic composite filler (B) will be described. The polymerization initiator is not particularly limited, and known polymerization initiators such as a photopolymerization initiator, a chemical polymerization initiator and a thermal polymerization initiator can be used. Among these, it is preferable to use a thermal polymerization initiator since it is excellent in production efficiency of the organic-inorganic composite filler (B). As the thermal polymerization initiator, an organic peroxides such as benzoyl peroxide, an azo compound such as azobisisobutyronitrile and the like may be suitably used. These polymerization initiators can be used not only singly but also in a combination of plurality thereof, regardless of the polymerization manner or the polymerization method. The amount of the polymerization initiator to be added is not particularly limited, but is generally 0.1% by mass to 10% by mass based on 100% by mass of all of the polymerizable monomer (b-2) used in the production of the organic-inorganic composite filler (B).

Next, the method for manufacturing the organic-inorganic composite filler (B) will be described by taking as an example a case in which a thermal polymerization initiator is used. The organic-inorganic composite filler (B) is prepared through the following main steps of (Step 1) to (Step 4).

(Step 1): a step of mixing the components constituting the (el) organic-inorganic composite filler such as a polymerizable monomer, a thermal polymerization initiator and an inorganic filler to obtain a mixture.
(Step 2): a step of applying heat to the mixture to polymerize the polymerizable monomer to obtain a cured product.
(Step 3): a step of pulverizing the cured product as necessary to obtain an organic-inorganic composite filler.
(Step 4): a step of performing a surface treatment on the organic-inorganic composite filler as necessary.

In the composite resin layer, the ground organic-inorganic composite filler obtained in the (Step 3) may be used as is, and the surface-treated organic-inorganic composite filler obtained in the (Step 4) may be used. Furthermore, in a case in which it is not in the form of a lump but is already in the form of fine particles at the stage of (Step 2), it may be used as it is as the organic-inorganic composite filler. Furthermore, the organic-inorganic composite filler may be used after subjecting a surface treatment in (Step 4).

Examples of the step of obtaining a mixture of the components in (Step 1) include a method of mixing the components such as a polymerizable monomer, a thermal polymerization initiator and an inorganic filler using a kneader, a method of aggregating an inorganic filler to obtain aggregated fillers having pores and a size of several μm to several tens of μm, immersing the aggregated fillers in a solution in which a thermal polymerization initiator and a polymerizable monomer are dissolved in an organic solvent to obtain a slurry, and then removing the organic solvent at a low temperature under reduced pressure to allow the polymerizable monomer to penetrate and cover the inside and surface of the aggregated filler, thereby mixing the components, and a method of press-molding an inorganic filler to obtain an inorganic filler molded body, immersing the molded body in a polymerizable monomer containing a thermal polymerization initiator and allowing the polymerizable monomer to penetrate into the inside of the molded body, thereby mixing the components, but are not limited thereto. In this step, by dissolving a surface treatment agent such as the above described organosilicon compound in the polymerizable monomer, the surface treatment of the inorganic filler and the mixing of the various components can be carried out simultaneously. This makes it possible to omit the step of surface treating the inorganic filler before mixing the various components.

In the step of obtaining a cured product in (Step 2), the polymerization temperature and polymerization time may be appropriately adjusted depending on the properties of the used thermal polymerization initiator and based on the discoloration of the organic-inorganic composite filler due to heat and the amount of residual unpolymerized monomer, but the polymerization temperature is generally 70° C. or higher and 150° C. or lower, and the polymerization time is several minutes to several hours. Depending on the polymerization method, polymerization conditions can be appropriately selected, such as polymerization in air, polymerization in an inert gas atmosphere such as nitrogen or argon, polymerization under normal pressure, or polymerization under pressure.

In the step of obtaining the organic-inorganic composite filler by pulverization in (Step 3), the pulverization method is not particularly limited, and may be either a wet method or a dry method. Specific examples of the pulverizer used for pulverization include a high speed rotating mill such as a hammer mill and a turbo-mill, a container driving type mill such as a ball mill, a planetary mill and a vibration mill, a medium stirring mill such as an attritor and a bead mill and a jet mill and the like, but are not limited thereto. The average particle diameter of the organic-inorganic composite filler (B) is not particularly limited, and the particle diameter can be appropriately adjusted depending on the desired property to be imparted to the composite resin layer. However, the average particle diameter is preferably 1 μm or more and 100 μm or less, and more preferably 10 μm or more and 30 μm or less. In an organic-inorganic composite filler having an average particle diameter of less than 1 μm, because a long time for pulverization is required for manufacturing, there is a case where discoloration is caused on the organic-inorganic composite filler itself to adversely affect the color tone of the composite resin layer. In the case of exceeding 100 μm, there is a case where the compressive strength of the composite resin layer decreases. The curable composition of the present disclosure may contain, as the organic-inorganic composite filler (B), only an organic-inorganic composite filler having an average particle diameter of 1 μm or more and 100 μm or less.

(Step 4) In the step of subjecting the organic-inorganic composite filler to a surface treatment, the same surface treatment agent as that which can be used for the surface treatment of the inorganic filler described above can be used. As for the surface treatment method, a known method can be applied, similar to the surface treatment of the inorganic filler. Furthermore, the amount of the surface treatment agent with respect to the organic-inorganic composite filler when performing the surface treatment is not particularly limited and may be appropriately adjusted depending on the particle diameter of the organic-inorganic composite filler and the like, but it is preferably 0.1 parts by mass or more and 5 parts by mass or less with respect to 100 parts by mass of the organic-inorganic composite filler.

There are no particular limitations on the content of the organic-inorganic composite filler (B), but is preferably within the range of 30% by mass or more and 60% by mass or less, and more preferably within the range of 35% by mass or more and 55% by mass or less, in the curable composition of the present disclosure. When the content of the organic-inorganic composite filler (B) is less than 30% by mass, there is a case where the brittleness of the composite resin layer increases and the fracture resistance decreases. When the content of the organic-inorganic composite filler (B) is more than 60% by mass, there is a case where the surface hardness, the abrasion resistance, the compressive strength and the like decrease.

The inorganic fine particle (C) that can be used in the curable composition of the present disclosure is not particularly limited in terms of its constituent elements, and any known inorganic filler can be used. Specific examples include silicon silica, alumina, titania, silica-titania, silica-titania-barium oxide, silica-zirconia, silica-alumina, lanthanum glass, borosilicate glass, soda glass, barium glass, strontium glass, glass ceramic, aluminosilicate glass, barium boroaluminosilicate glass, strontium boroaluminosilicate glass, fluoroaluminosilicate glass, calcium fluoroaluminosilicate glass, strontium fluoroaluminosilicate glass, barium fluoroaluminosilicate glass, strontium calcium fluoroaluminosilicate glass and the like.

A shape of these of the inorganic fine particle (C) is not particularly limited, and may be any shape such as spherical, needle-like, plate-like, ground-like and scaly-shapes, and aggregate thereof may be used without any problems. The above described inorganic fine particle (C) is not limited to these and may be used alone or in a combination of plurality thereof.

In the present disclosure, the average particle diameter of all of the inorganic fine particle (C) contained in the curable composition must be 1 μm or less in order to exhibit good fracture resistance, excellent polishing property, and surface lubricative property in the composite resin layer. When the average particle diameter of the inorganic fine particle (C) exceeds 1 μm, the fracture resistance of the composite resin layer decreases. In addition, because the polishing property decreases, a smooth surface cannot be obtained therefore discoloration easily occurs. In the present disclosure, an inorganic particle having an average particle diameter of more than 1 μm may be contained in an amount that does not impair the effects of the present disclosure. The amount of an inorganic particle having an average particle diameter of more than 1 μm that does not impair the effects of the present disclosure may be less than 1 mass %, less than 0.5 mass %, less than 0.1 mass %, less than 0.05 mass %, less than 0.01 mass %, or less than 0.001 mass % in the curable composition of the present disclosure. In the present disclosure, an inorganic particle having an average particle diameter exceeding 1 μm may not be contained. In addition, in the present disclosure, an inorganic particle having a particle diameter exceeding 1 μm may not be contained.

The inorganic fine particle (C) may be surface-treated with a surface treatment agent or the like. Specific examples of the surface treatment agent include a surfactant, an organic acid, an inorganic acid, an organosilicon compound, an organozirconium compound, an organotitanium compound, an organoaluminum compound, a metal alkoxide compound and the like, and an organosilicon compound is the most commonly used. As the organosilicon compound, the same organosilicon compounds that can be used for the surface treatment of the above described inorganic filler (b-1) can be used. Specific examples of the surface treatment method include a method of spraying the surface treatment agent in the state of allowing the inorganic fine particle to flow, and a method of dispersing the inorganic fine particle in a solution including the surface treatment agent. The surface treatment agent and the surface treatment method are not limited to those described above, and each of them can be used alone or in a combination of plurality thereof. Furthermore, the amount of the surface treatment agent relative to the inorganic fine particle (C) when performing the surface treatment is not particularly limited, and may be appropriately adjusted depending on the particle diameter of the inorganic fine particle (C) and the like.

There are no particular limitations on the content of the inorganic fine particle (C), but is preferably within the range of 10% by mass or more and 50% by mass or less, and more preferably within the range of 15% by mass or more and 35% by mass or less, in the curable composition of the present disclosure. When the content of the inorganic fine particle (C) is less than 10% by mass, there is a case where the surface hardness, the abrasion resistance, the compressive strength and the like of the composite resin layer decrease. When the content of the inorganic fine particle (C) is more than 50% by mass, there is a case where the brittleness of the composite resin layer increases and the fracture resistance decreases.

As the non-crosslinked polymer particle (D) that can be used in the curable composition of the present disclosure, a particle of a homopolymer of the above described monofunctional polymerizable monomer having (meth)acryloyloxy group or (meth)acrylamide group, a particle of a copolymer combining two or more types, and a particle of a copolymer combining a monofunctional polymerizable monomer having a (meth)acryloyloxy group or a (meth)acrylamide group with other monofunctional polymerizable monomer such as styrene, α-methylstyrene, isoprene, butadiene, isobutylene, vinyl acetate, vinyl chloride, vinyl alcohol, ethylene, propylene, maleic acid, itaconic acid, and maleic anhydride, and the like, can be used without any limitations. Furthermore, there is no problem in using a particle of a homopolymer of the above described other monofunctional polymerizable monomer, or a particle of a copolymer of two or more kinds of monomers. The copolymer particle may be any copolymer, such as a random copolymer, an alternating copolymer or a block copolymer.

Among these of the non-crosslinked polymer particle (D), it is preferable to use a particle of a homopolymer of a monofunctional polymerizable monomer having a (meth)acryloyloxy group, which is the most commonly used dental material, or a particle of a particle of a copolymer combining two or more types. Specific examples of the non-crosslinked polymer particle (D) include a particle of homopolymer such as methyl (meth)acrylate, ethyl (meth)acrylate, propyl (meth)acrylate, isopropyl (meth)acrylate, isobutyl (meth)acrylate, butyl (meth)acrylate and 2-ethylhexyl (meth)acrylate, and a particle of a copolymer combining two or more types, but are not limited thereto. These of the non-crosslinked polymer particle (D) can be used alone or in a combination of plurality thereof. As the non-crosslinked polymer particle (D), it is more preferable to use polymethyl methacrylate particle or a copolymer particle of methyl methacrylate and ethyl methacrylate, and it is most preferable to use polymethyl methacrylate particle. By using these of the non-crosslinked polymer particle (D), it is possible to improve the fracture resistance and compressive strength of the composite resin layer in a well-balanced manner. The curable composition of the present disclosure may contain, as the non-crosslinked polymer particle (D), only a particle of a homopolymer of a monofunctional polymerizable monomer having a (meth)acryloyloxy group and/or a particle of a copolymer combining two or more types, may contain only polymethyl methacrylate particle and/or a copolymer particle of methyl methacrylate and ethyl methacrylate, or may contain only polymethyl methacrylate particle.

The polymerization method for manufacturing these of the non-crosslinked polymer particle (D) is not particularly limited, and any polymerization method such as emulsion polymerization or suspension polymerization may be used without any problem. The shape of these of the non-crosslinked polymer particle (D) may be any shape, such as spherical, crushed, hollow and the like without any limitation, but the spherical shape is preferable. The weight average molecular weight of the non-crosslinked polymer particle (D) is not particularly limited, but the weight average molecular weight is preferably 1 μm or more and is more preferably within a range of 150,000 to 1,500,000. Herein, the weight average molecular weight means the average molecular weight which is calculated based on molecular weight distribution measured by gel permeation chromatography. The curable composition of the present disclosure may contain, as the non-crosslinked polymer particle (D), only non-crosslinked polymer particle having a weight average molecular weight of 10,000 or more and 2,000,000 or less, only non-crosslinked polymer particle having a weight average molecular weight of 50,000 or more and 1,500,000 or less, or only non-crosslinked polymer particle having a weight average molecular weight of 100,000 or more and 1,500,000 or less.

The average particle diameter of the non-crosslinked polymer particle (D) is preferably with in a range of 1 μm to 100 μm, more preferably within a range of 1 μm to 80 μm, and further more preferably within a range of 5 μm to 50 μm. When the average particle diameter of the non-crosslinked polymer particle (D) is less than 1 μm, there is a case where the compressive strength of the composite resin layer decreases. When the average particle diameter of the non-crosslinked polymer particle (D) exceeds 100 μm, there is a case where the adhesive property of the composite resin layer to the denture base decreases. The curable composition of the present disclosure may contain, as the non-crosslinked polymer particle (D), only non-crosslinked polymer particle having an average particle diameter of 1 μm or more and 100 μm or less, only non-crosslinked polymer particle having an average particle diameter of 3 μm or more and 80 μm or less, or only non-crosslinked polymer particle having an average particle diameter of 5 μm or more and 50 μm or less. The curable composition of the present disclosure may contain, as the non-crosslinked polymer particle (D), only non-crosslinked polymer particle having a weight average molecular weight of 10,000 or more and 2,000,000 or less and an average particle diameter of 1 μm or more and 100 μm or less.

The content of the non-crosslinked polymer particles (D) in the curable composition of the present disclosure must be within a range of 1% by mass or more and 5% by mass or less. When the content of the non-crosslinked polymer particles (D) is less than 1% by mass, there is a case where the fracture resistance of the composite resin layer and the adhesive property to the denture base decrease. When the content of the non-crosslinked polymer particles (D) is more than 5% by mass, there is a case where the surface hardness, the abrasion resistance, the compressive strength and the like of the composite resin layer decrease.

The curable composition of the present disclosure preferably contains a polymerization initiator. As the type of the polymerization initiator, there are those which initiate radical polymerization by heating (thermal polymerization initiator), those which initiate radical polymerization by the action of compounds of two or more components such as redox initiator (in the dental field, called sometimes as “chemical polymerization initiator”, and hereinafter referred to as “chemical polymerization initiator”.) and those which initiate radical polymerization by irradiation with light (photopolymerization initiators), and any of these polymerization initiators can be used in the present disclosure without any limitations. However, it is preferable to use a thermal polymerization initiator, since it is easy to mold the composite resin layer and is easy obtain high mechanical property.

Specific examples of the thermal polymerization initiator include an organic peroxide such as benzoyl peroxide, p-chlorobenzoyl peroxide, 2,4-dichlorobenzoyl peroxide, acetyl peroxide, lauroyl peroxide, tertiary butyl peroxide, cumene hydroperoxide, 2,5-dimethyl-2,5-di (benzoylperoxy) hexane, 2,5-dihydroperoxide, methyl ethyl ketone peroxide and tertiary butyl peroxybenzoate, an azo compound such azobisisobutyronitrile, azobisisobutyric acid methyl, or azobiscyanovaleric, but it is not limited thereto. These thermal polymerization initiators may be used alone or in a combination of plurality thereof. Among these thermal polymerization initiators, it is most preferable to use benzoyl peroxide and/or azobisisobutyronitrile.

Specific examples of the chemical polymerization initiator include organic peroxide/amine compound, organic peroxide/amine compound/sulfinic acid salt, or organic peroxide/amine compound/borate compound, but are not limited thereto. These chemical polymerization initiator may be used alone or in a combination of a plurality thereof.

Specific examples of the photopolymerization initiator include α-diketones, benzophenones, acylphosphine oxides, α-aminoacetophenones, ketals, coumarins, titanocenes and the like, but are not limited thereto. Specific examples of the photopolymerization accelerator include tertiary amines, triazine compounds, diaryliodonium salts, tin compounds, aldehyde compounds and sulfur-containing compounds, but are not limited thereto. These photopolymerization initiators and photopolymerization accelerators can be used alone or in a combination of plurality thereof.

There are no particular limitations on the content of the polymerization initiator, but is preferably within the range of 0.1% by mass or more and 1.5% by mass or less, and more preferably within the range of 35% by mass or more and 55% by mass or less, with respect to 100 parts by mass of the total content of the polymerizable monomer (A) and the polymerization initiator. When the content of the polymerization initiator is less than 0.1% by mass, there is a case where the polymerization of the curable composition of the present disclosure is insufficient and various mechanical properties of the composite resin layer is reduced or the composite resin layer is easily colored. When the content of the polymerization initiator is more than 1.5% by mass, there is a case where the composite resin layer becomes discolored.

Besides the components (A) to (D), components such as an ultraviolet absorber such as 2-hydroxy-4-methylbenzophenone, a polymerization inhibitor such as hydroquinone, hydroquinone monomethyl ether and 2,5-ditertiarybutyl-4-methylphenol, a chain transfer agent, a discoloration inhibitor, an antibacterial agent, a coloring pigment, and other additives known in the art may be added as necessary and as desired to the curable composition of the present disclosure.

The composite resin tooth of the present disclosure may have a single layer structure or a layer structure of two or more layers. In the case of having a layer structure of two or more layers, at least one of the layers is a composite resin layer formed by polymerizing and curing the curable composition of the present disclosure. The composite resin tooth of the present disclosure may have a single-layer structure consisting of only a composite resin layer, or may have a layer structure consisting of only two or more composite resin layers. There are no particular limitations on the material constituting the layer other than the composite resin layer, but it is preferable that the layer is an acrylic resin layer in which the main component is polymethyl methacrylate, which is formed by polymerizing and curing the mixture prepared by mixing a liquid material in which the main component is methylmethacrylate and a powder material in which the main component is polymethylmethacrylate, as with conventional composite resin tooth. In addition, in the case of having a layer structure of two or more layers, it is preferable that the outermost layer including the labial surface is a composite resin layer in the case of an anterior tooth and the outermost layer including the occlusal surface is a composite resin layer in the case of a molar tooth. This reduces the wear rate of the composite resin tooth of the present disclosure in the oral cavity.

There are no particular limitations on the shape and size of the composite resin tooth of the present disclosure, as well as the shape and size of each layer, and there is no problem if it has retention holes to ensure a mechanical fit with the denture base.

The method for manufacturing the composite resin tooth of the present disclosure is not particularly limited, and the composite resin tooth of the present disclosure can be manufactured by methods such as compression molding, injection molding, and injection compression molding, but is not limited thereto.

### EXAMPLES

Hereinafter, the present disclosure will be described in detail with reference to Examples and Comparative Examples. However, the present disclosure is not limited to these Examples. The various components used for preparing the curable compositions of Examples and Comparative Examples and their abbreviations are as follows.

### [Polymerizable Monomer (A)]

UDMA: urethane dimethacrylate
UDA: 1.6 bis [(2-phenoxy-2′-acryloxy) isopropyl-oxy-carbonylamino] hexane
Bis-GMA: 2,2-bis [4-[2-hydroxy-3-(methacryloyloxy) propyloxy] phenyl] propane
TEGDMA: triethylene glycol dimethacrylate
MMA: methyl methacrylate

### [Organic-Inorganic Composite Filler (B)]

O1: organic-inorganic composite filler 1 (average particle diameter: 23 μm, content of inorganic filler (b-1): 18% by mass, average particle diameter of inorganic filler (b-1): 16 nm)
O2: organic-inorganic composite filler 2 (average particle diameter: 20 μm, content of inorganic filler (b-1): 32.5% by mass, average particle diameter of inorganic filler (b-1): 9 nm)
O3: organic-inorganic composite filler 3 (average particle diameter: 28 μm, content of inorganic filler (b-1): 50% by mass, average particle diameter of inorganic filler (b-1): 16 nm)
O4: organic-inorganic composite filler 4 (average particle diameter: 20 μm, content of inorganic filler (b-1): 12% by mass, average particle diameter of inorganic filler (b-1): 16 nm)
O5: organic-inorganic composite filler 5 (average particle diameter: 25 μm, content of inorganic filler (b-1): 35% by mass, average particle diameter of inorganic filler (b-1): 16 nm)
O6: organic-inorganic composite filler 6 (average particle diameter: 30 μm, content of inorganic filler (b-1): 10% by mass, average particle diameter of inorganic filler (b-1): 16 nm)
O7: organic-inorganic composite filler 7 (average particle diameter: 55 μm, content of inorganic filler (b-1): 75% by mass, average particle diameter of inorganic filler (b-1): 4 nm)
O8: organic-inorganic composite filler 8 (average particle diameter: 22 μm, content of inorganic filler (b-1): 50% by mass, average particle diameter of inorganic filler (b-1): 3 nm)

### [Inorganic Fine Particle (C) Having an Average Particle Diameter of 1 μm or Less]

I1: fumed silica (Aerosil OX-50 (manufactured by Evonik Industries), average particle diameter: 40 nm)
I2: fumed silica (Aerosil R-972 (manufactured by Evonik Industries), average particle dimeter: 16 nm)

### [Other Inorganic Fine Particle (C′)]

I′1: Spherical silica (average particle dimeter: 3 μm)
I′2: crushed silica (average particle dimeter: 1.5 μm)

### [Non-Crosslinked Polymer Particle (D)]

nCP1: polymethyl methacrylate (average particle diameter: 8 μm, weight average molecular weight: approximately 800,000, shape: spherical)
nCP2: polymethyl methacrylate (average particle diameter: 50 μm, weight average molecular weight: approximately 1,000,000, shape: spherical)
nCP3: polymethyl methacrylate (average particle diameter: 80 μm, weight average molecular weight: approximately 1,000,000, shape: spherical)
nCP4: polymethyl methacrylate (average particle diameter: 4 μm, weight average molecular weight: approximately 800,000, shape: spherical)
nCP5: copolymer of methyl methacrylate (MMA) and ethyl methacrylate (EMA) (MMA/EMA=70/30) (average particle diameter: 65 μm, weight average molecular weight: approximately 350,000, shape: spherical)
nCP6: polyethyl methacrylate (average particle diameter: 4 μm, weight average molecular weight: approximately 40,000, shape: spherical)
nCP7: polymethyl methacrylate (average particle diameter: 1.5 μm, weight average molecular weight: approximately 150,000, shape: spherical)
nCP8: polymethyl methacrylate (average particle diameter: 0.4 μm, weight average molecular weight: approximately 1,500,000, shape: spherical)
nCP9: polymethyl methacrylate (average particle diameter: 120 μm, weight average molecular weight: approximately 1,600,000, shape: spherical)
nCP10: polymethyl methacrylate (average particle diameter: 5 μm, weight average molecular weight: approximately 300,000, shape: spherical)
nCP11: polymethyl methacrylate (average particle diameter: 100 μm, weight average molecular weight: approximately 1,000,000, shape: spherical)

### [Crosslinked Polymer Particle (D′)]

CP1: Crosslinked polymethyl methacrylate (average particle diameter: 2.2 μm, shape: spherical)
CP2: Crosslinked polyurethane (average particle diameter: 6 μm, shape: spherical)

### [Polymerization Initiator]

BPO: Benzoyl peroxide

### [Manufacture of Organic-Inorganic Composite Filler (B)]

### [Manufacture of Organic-Inorganic Composite Filler 1 (O1)]

A resin mixture was prepared by mixing 80 parts by mass of UDMA, 20 parts by mass of ethylene glycol dimethacrylate and 0.3 parts by mass of BPO. After kneading 82 parts by mass of the resin mixture and 18 parts by mass of Aerosil R972 until homogeneous, the kneaded material was heated at 100° C. for 4 hours in a nitrogen atmosphere to obtain a cured product. The obtained cured product was pulverized until the average particle diameter became 23 μm to obtain the organic-inorganic composite filler 1 (O1). The average particle diameter was measured by a laser diffraction type grain size measuring apparatus (Microtrac MT3300EXII, manufactured by Microtrac Bell Co., Ltd.).

### [Manufacture of Organic-Inorganic Composite Filler 2 (O2)]

A resin mixture was prepared by mixing 80 parts by mass of UDMA, 20 parts by mass of ethylene glycol dimethacrylate and 0.5 parts by mass of BPO. After kneading 67.5 parts by mass of the resin mixture and 32.5 parts by mass of Aerosil R972 until homogeneous, the kneaded material was heated at 100° C. for 4 hours in a nitrogen atmosphere to obtain a cured product. The obtained cured product was pulverized until the average particle diameter became 20 μm to obtain the organic-inorganic composite filler 2 (O2). The average particle diameter was measured by a laser diffraction type grain size measuring apparatus (Microtrac MT3300EXII, manufactured by Microtrac Bell Co., Ltd.).

### [Manufacture of Organic-Inorganic Composite Filler 3 (O3)]

A resin mixture was prepared by mixing 50 parts by mass of UDMA, 50 parts by mass of neopentyl glycol dimethacrylate and 1.0 parts by mass of BPO. After kneading 50 parts by mass of the resin mixture and 50 parts by mass of Aerosil R972 until homogeneous, the kneaded material was heated at 100° C. for 4 hours in a nitrogen atmosphere to obtain a cured product. The obtained cured product was pulverized until the average particle diameter became 28 μm to obtain the organic-inorganic composite filler 3 (O3). The average particle diameter was measured by a laser diffraction type grain size measuring apparatus (Microtrac MT3300EXII, manufactured by Microtrac Bell Co., Ltd.).

### [Manufacture of Organic-Inorganic Composite Filler 4 (O4)]

A resin mixture was prepared by mixing 80 parts by mass of UDMA, 20 parts by mass of ethylene glycol dimethacrylate and 0.3 parts by mass of BPO. After kneading 88 parts by mass of the resin mixture and 12 parts by mass of Aerosil R972 until homogeneous, the kneaded material was heated at 100° C. for 4 hours in a nitrogen atmosphere to obtain a cured product. The obtained cured product was pulverized until the average particle diameter became 20 μm to obtain the organic-inorganic composite filler 4 (O4). The average particle diameter was measured by a laser diffraction type grain size measuring apparatus (Microtrac MT3300EXII, manufactured by Microtrac Bell Co., Ltd.).

### [Manufacture of Organic-Inorganic Composite Filler 5 (O5)]

A resin mixture was prepared by mixing 80 parts by mass of UDMA, 20 parts by mass of ethylene glycol dimethacrylate and 0.3 parts by mass of BPO. After kneading 65 parts by mass of the resin mixture and 35 parts by mass of Aerosil R972 until homogeneous, the kneaded material was heated at 100° C. for 4 hours in a nitrogen atmosphere to obtain a cured product. The obtained cured product was pulverized until the average particle diameter became 25 μm to obtain the organic-inorganic composite filler 5 (O5). The average particle diameter was measured by a laser diffraction type grain size measuring apparatus (Microtrac MT3300EXII, manufactured by Microtrac Bell Co., Ltd.).

### [Manufacture of Organic-Inorganic Composite Filler 6 (O6)]

A resin mixture was prepared by mixing 80 parts by mass of UDMA, 20 parts by mass of ethylene glycol dimethacrylate and 0.3 parts by mass of BPO. After kneading 90 parts by mass of the resin mixture and 10 parts by mass of Aerosil R972 until homogeneous, the kneaded material was heated at 100° C. for 4 hours in a nitrogen atmosphere to obtain a cured product. The obtained cured product was pulverized until the average particle diameter became 30 μm to obtain the organic-inorganic composite filler 6 (O6). The average particle diameter was measured by a laser diffraction type grain size measuring apparatus (Microtrac MT3300EXII, manufactured by Microtrac Bell Co., Ltd.).

### [Manufacture of Organic-Inorganic Composite Filler 7 (O7)]

A surface treatment liquid (total mass: 11.5 parts by mass) was prepared by mixing 3.0 part by mass of γ-methacryloyloxypropyl trimethoxysilane, 0.5 part by mass of ion-exchanged water and 8.0 parts by mass of absolute ethanol. Next, various raw materials: silica, alumina, aluminum phosphate, sodium fluoride, and strontium carbonate (glass composition: SiO₂: 26.4% by mass, Al₂O₃: 29.3% by mass, SrO: 20.5% by mass, P₂O₅: 10.9% by mass, Na₂O: 2.5% by mass, and F: 10.4% by mass) were mixed and the mixed material was molten at 1400° C. in a melting furnace. The molten liquid was taken out from the melting furnace and was quenched in water to manufacture a fluoroaluminosilicate glass. The resulting fluoroaluminosilicate glass was pulverized until the 50% particle diameter (D50) became 4 μm to obtain acid-reactive glass powder. Thereafter, the above described surface treatment liquid and 100 parts by mass of the above described fluoroaluminosilicate glass powder were dry-mixed then heat-treated at 110° C. for 5 hours using a hot air dryer to obtain a surface-treated glass powder. Further, a resin mixture was prepared by mixing 50 parts by mass of Bis-GMA, 50 parts by mass of triethylene glycol dimethacrylate and 0.2 parts by mass of BPO. After kneading 25 parts by mass of the resin mixture and 75 parts by mass of the surface-treated glass powder until homogeneous, the kneaded material was heated at 100° C. for 4 hours in a nitrogen atmosphere to obtain a cured product. The obtained cured product was pulverized until the 50% particle diameter (D50) became 55 μm to obtain the organic-inorganic composite filler 7 (O7). The 50% particle diameter was measured by a laser diffraction type grain size measuring apparatus (Microtrac MT3300EXII, manufactured by Microtrac Bell Co., Ltd.).

### [Manufacture of Organic-Inorganic Composite Filler 8 (O8)]

A surface treatment liquid (total mass: 11.5 parts by mass) was prepared by mixing 3.0 part by mass of γ-methacryloyloxypropyl trimethoxysilane, 0.5 part by mass of ion-exchanged water and 8.0 parts by mass of absolute ethanol. Next, various raw materials: silica, alumina, aluminum phosphate, sodium fluoride, and strontium carbonate (glass composition: SiO₂: 26.4% by mass, Al₂O₃: 29.3% by mass, SrO: 20.5% by mass, P₂O₅: 10.9% by mass, Na₂O: 2.5% by mass, and F: 10.4% by mass) were mixed and the mixed material was molten at 1400° C. in a melting furnace. The molten liquid was taken out from the melting furnace and was quenched in water to manufacture a fluoroaluminosilicate glass. The resulting fluoroaluminosilicate glass was pulverized until the 50% particle diameter (D50) became 3 μm to obtain acid-reactive glass powder. Thereafter, the above described surface treatment liquid and 100 parts by mass of the above described fluoroaluminosilicate glass powder were dry-mixed then heat-treated at 110° C. for 5 hours using a hot air dryer to obtain a surface-treated glass powder. Further, a resin mixture was prepared by mixing 50 parts by mass of Bis-GMA, 50 parts by mass of triethylene glycol dimethacrylate and 0.2 parts by mass of BPO. After kneading 50 parts by mass of the resin mixture and 50 parts by mass of the surface-treated glass powder until homogeneous, the kneaded material was heated at 100° C. for 4 hours in a nitrogen atmosphere to obtain a cured product. The obtained cured product was pulverized until the 50% particle diameter (D50) became 40 μm to obtain the organic-inorganic composite filler 8 (O8). The 50% particle diameter was measured by a laser diffraction type grain size measuring apparatus (Microtrac MT3300EXII, manufactured by Microtrac Bell Co., Ltd.).

The above described various components were mixed in the ratios shown in Tables 1 to 3 to prepare the curable compositions of the Examples and Comparative Examples. The prepared curable compositions were evaluated for surface hardness, compressive strength, compressive displacement and adhesive property to denture base material according to the test methods described below. In the present specification, the amount of compressive displacement is used as an index of the fracture resistance of a material, and it is determined that the greater the amount of compressive displacement, the more excellent the fracture resistance.

### [Surface Hardness]

In accordance with ISO 6507-1:2018, surface hardness was measured using the following procedure. Each curable composition was filled into a mold (Φ25 mm, thickness 2.5 mm), and then pressurized and heated molding (press pressure: 3t, molding temperature: 120° C., pressing time 5 minutes) was performed. The prepared cured product was polished to a thickness of 2 mm or more, and used as a test specimen. The surface hardness of each test specimen was measured under conditions of 23±5° C. and HV0.2 using a micro Vickers hardness tester HM-102 (manufactured by Mitsutoyo Corporation). As a result, when the surface hardness was 30 or more, it was determined that the surface hardness was excellent.

### [Compressive Strength and Compressive Displacement]

In accordance with JIS T 6603:1994, the compressive strength and compressive displacement were measured according to the following procedure. Each curable composition was filled into a mold (Φ6 mm, height 12 mm), and then pressurized and heated molding (press pressure: 3t, molding temperature: 120° C., pressing time 5 minutes) was performed. The prepared cured product was immersed in water at 37° C. for 24 hours to prepare a test specimen. The compressive strength and compressive displacement of each specimen were measured using an Instron universal testing machine (model: 5567A) at a crosshead speed of 1 mm/min. The compressive strength and the amount of compressive displacement were evaluated according to the following evaluation criteria. When the evaluation criteria were A or B, the specimen was determined to have good compressive strength and fracture resistance, respectively.

### <Evaluation Criteria>

### <<Compressive Strength>>

A: 500 MPa or more
B: 430 MPa or more and less than 500 MPa
C: less than 430 MPa

### <<Compression Displacement Amount>>

A: 4.0 mm or more
B: 3.5 mm or more and less than 4.0 mm
C: less than 3.5 mm

### [Adhesive Property to Denture Base Material]

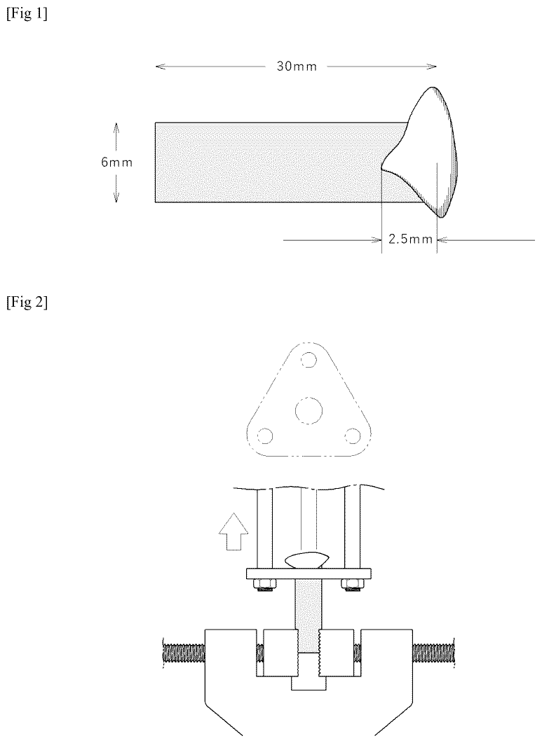

In accordance with ISO 22112:2017, adhesive property to the denture base material was evaluated using the following procedure. Each curable composition was filled into a mold having the outer shape of an upper anterior tooth of the NC Veracia Anterior (manufactured by SHOFU INC.), and then pressurized and heated molding (press pressure: 3t, molding temperature: 120° C., pressing time 5 minutes) was performed to manufacture an artificial tooth. A wax having a size of 30×10×6 (mm) was prepared, and the 10×6 mm surface was melted using a hot plate or the like. The central part of the lingual side of the artificial tooth was then pressed into melted surface by about 2.5 mm, and the artificial tooth was fixed to the wax by holding until the wax cooled. It was embedded in plaster using a dental flask, and the wax was then washed off with boiling water. The area from which the wax had been removed was filled with denture base material “SHOFU URBAN 8S color” (manufactured by SHOFU INC.), a clamp was attached to the flask, the denture base material was cured by immersing in 70° C. water for 90 minutes, and then in boiling water for 30 minutes to use as the test specimen (FIG. 1). As shown in FIG. 2, the test specimen was hooked on the lingual surface of the artificial tooth to a special jig, and the end of the denture base material was fixed with a clamp. Thereafter, a load was applied in the tensile direction to the adhesive interface at a displacement rate of 1 mm/min, causing fracture at the adhesive site of the test specimen. An Instron universal testing machine (model: 5567A) was used to apply a tensile load to the test specimen. Ten specimens were tested for each curable composition, and the failure mode of each specimen was observed. When evaluated according to the following criteria, a sample was determined to have good adhesive property to denture base material in the case that rating is A or B.

### <Evaluation Criteria>

A: cohesive failure exhibited in 10 or 9 out of 10 specimens.
B: cohesive failure exhibited in 8 or 7 out of 10 specimens.
C: cohesive failure exhibited in 6 or less out of 10 specimens.

The results of evaluation of the curable compositions of the Examples and Comparative Examples are shown in Tables 1 to 4.

### Curable Compositions (Mass %) Used in Examples and Evaluation Results

TABLE 1

| | | Exam- | Exam- | Exam- | Exam- | Exam- | Exam- | Exam- | Exam- | Exam- | Exam- |
|---|---|---|---|---|---|---|---|---|---|---|---|
| | | ple 1 | ple 2 | ple 3 | ple 4 | ple 5 | ple 6 | ple 7 | ple 8 | ple 9 | ple 10 |
| Polymerizable | UDMA | 21.0 | — | 21.0 | — | 20.1 | — | — | — | 21.0 | 21.0 |
| monomer | UDA | — | 21.0 | — | — | — | 24.2 | — | — | — | — |
| (A) | Bis-GMA | — | — | — | 21.0 | — | — | 22.8 | 22.8 | — | — |
| | TEGDMA | 9.0 | 9.0 | 7.5 | 9.0 | 8.8 | 10.4 | 9.8 | 9.8 | 9.0 | 9.0 |
| | MMA | — | — | 1.5 | — | — | — | — | — | — | — |
| | Amount of | — | — | 5.0 | — | — | — | — | — | — | — |
| | MMA in (A) | | | | | | | | | | |
| Organic- | O1 | 45.0 | 45.0 | 45.0 | 45.0 | 40.0 | 45.0 | — | — | — | 45.0 |
| inorganic | O2 | — | — | — | — | — | — | 42.5 | — | — | — |
| composite | O3 | — | — | — | — | — | — | — | — | — | — |
| filler | O4 | — | — | — | — | — | — | — | 42.5 | — | — |
| (B) | O5 | — | — | — | — | — | — | — | — | 45.0 | — |
| | O6 | — | — | — | — | — | — | — | — | — | — |
| | O7 | — | — | — | — | — | — | — | — | — | — |
| | O8 | — | — | — | — | — | — | — | — | — | — |
| Inorganic fine | I1 | 3.7 | 3.7 | 3.7 | 3.7 | 5.0 | — | 3.7 | 3.7 | 3.7 | 3.7 |
| particle (C) | I2 | 18.0 | 18.0 | 18.0 | 18.0 | 25.0 | 15.0 | 18.0 | 18.0 | 18.0 | 18.0 |
| Other inorganic | I′1 | — | — | — | — | — | — | — | — | — | — |
| fine particle (C′) | I′2 | — | — | — | — | — | — | — | — | — | — |
| Non- | nCP1 | 3.0 | 3.0 | 3.0 | 3.0 | 1.0 | 5.0 | 3.0 | 3.0 | 3.0 | — |
| crosslinked | nCP2 | — | — | — | — | — | — | — | — | — | 3.0 |
| polymer | nCP3 | — | — | — | — | — | — | — | — | — | — |
| particle (D) | nCP4 | — | — | — | — | — | — | — | — | — | — |
| | nCP5 | — | — | — | — | — | — | — | — | — | — |
| | nCP6 | — | — | — | — | — | — | — | — | — | — |
| | nCP7 | — | — | — | — | — | — | — | — | — | — |
| | nCP8 | — | — | — | — | — | — | — | — | — | — |
| | nCP9 | — | — | — | — | — | — | — | — | — | — |
| | nCP10 | — | — | — | — | — | — | — | — | — | — |
| | nCP11 | — | — | — | — | — | — | — | — | — | — |
| Crosslinked | CP1 | — | — | — | — | — | — | — | — | — | — |
| polymer particle (D′) | CP2 | — | — | — | — | — | — | — | — | — | — |
| Polymerization | BPO | 0.3 | 0.3 | 0.3 | 0.3 | 0.1 | 0.4 | 0.2 | 0.2 | 0.3 | 0.3 |
| Initiator | Polymerization | 1.0 | 1.0 | 1.0 | 1.0 | 0.3 | 1.1 | 0.6 | 0.6 | 1.0 | 1.0 |
| | Initiator/ | | | | | | | | | | |
| | ((A) + Polymerization | | | | | | | | | | |
| | Initiator) | | | | | | | | | | |
| | (mass %) | | | | | | | | | | |

| | | | | | | | | | | |
|---|---|---|---|---|---|---|---|---|---|---|
| Total | 100.0 | 100.0 | 100.0 | 100.0 | 100.0 | 100.0 | 100.0 | 100.0 | 100.0 | 100.0 |
| [Surface hardness](HV0.2) | 34 | 35 | 32 | 34 | 37 | 31 | 39 | 30 | 40 | 34 |
| Compressive strength (Mpa) | 529 | 521 | 514 | 532 | 528 | 509 | 529 | 502 | 539 | 512 |
| Evaluation | A | A | A | A | A | A | A | A | A | A |
| Compressive displacement (mm) | 4.3 | 4.3 | 4.5 | 4.2 | 4.1 | 4.0 | 4.1 | 4.5 | 4.0 | 4.1 |
| Evaluation | A | A | A | A | A | A | A | A | A | A |
| Adhesive property to denture base material | A | A | A | A | A | A | A | A | A | A |

### Curable Compositions (Mass %) Used in Examples and Evaluation Results

TABLE 2

| | | Exam- | Exam- | Exam- | Exam- | Exam- | Exam- | Exam- | Exam- | Exam- | Exam- |
|---|---|---|---|---|---|---|---|---|---|---|---|
| | | ple 11 | ple 12 | ple 13 | ple 14 | ple 15 | ple 16 | ple 17 | ple 18 | ple 19 | ple 20 |
| Polymerizable | UDMA | 21.0 | 21.0 | — | 20.8 | — | — | 21.0 | 19.6 | 21.0 | — |
| monomer | UDA | — | — | 23.6 | — | 21.0 | 21.0 | — | — | — | — |
| (A) | Bis-GMA | — | — | — | — | — | — | — | — | — | 32.0 |
| | TEGDMA | 9.0 | 9.0 | 10.0 | 8.9 | 9.0 | 9.0 | 6.0 | 4.2 | 9.0 | 8.0 |
| | MMA | — | — | — | — | — | — | 3.0 | 4.2 | — | — |
| | Amount of | — | — | — | — | — | — | 10.0 | 15.0 | — | — |
| | MMA in (A) | | | | | | | | | | |
| Organic- | O1 | 45.0 | 45.0 | 50.0 | 30.0 | — | — | 42.5 | 48.4 | 45.0 | — |
| inorganic | O2 | — | — | — | — | — | — | — | — | — | — |
| composite | O3 | — | — | — | — | 45.0 | — | — | — | — | — |
| filler | O4 | — | — | — | — | — | — | — | — | — | — |
| (B) | O5 | — | — | — | — | — | — | — | — | — | — |
| | O6 | — | — | — | — | — | 40.0 | — | — | — | — |
| | O7 | — | — | — | — | — | — | — | — | — | 30.0 |
| | O8 | — | — | — | — | — | — | — | — | — | — |
| Inorganic fine | I1 | 3.7 | 3.7 | — | 27.0 | 3.7 | 3.7 | 4.2 | 3.4 | 3.7 | 4.0 |
| particle (C) | I2 | 18.0 | 18.0 | 13.0 | 10.0 | 18.0 | 23.0 | 20.0 | 17.0 | 18.0 | 22.6 |
| Other inorganic | I′1 | — | — | — | — | — | — | — | — | — | — |
| fine particle (C′) | I′2 | — | — | — | — | — | — | — | — | — | — |
| Non- | nCP1 | — | — | 3.0 | 3.0 | 3.0 | 3.0 | 3.0 | — | — | 3.0 |
| crosslinked | nCP2 | — | — | — | — | — | — | — | — | — | — |
| polymer | nCP3 | 3.0 | — | — | — | — | — | — | — | — | — |
| particle (D) | nCP4 | — | 3.0 | — | — | — | — | — | — | — | — |
| | nCP5 | — | — | — | — | — | — | — | 3.0 | — | — |
| | nCP6 | — | — | — | — | — | — | — | — | 3.0 | |
| | nCP7 | — | — | — | — | — | — | — | — | — | — |
| | nCP8 | — | — | — | — | — | — | — | — | — | — |
| | nCP9 | — | — | — | — | — | — | — | — | — | — |
| | nCP10 | — | — | — | — | — | — | — | — | — | — |
| | nCP11 | — | — | — | — | — | — | — | — | — | — |
| Crosslinked | CP1 | — | — | — | — | — | — | — | — | — | — |
| polymer particle (D′) | CP2 | — | — | — | — | — | — | — | — | — | — |
| Polymerization | BPO | 0.3 | 0.3 | 0.4 | 0.3 | 0.3 | 0.3 | 0.3 | 0.2 | 0.3 | 0.4 |
| Initiator | Polymerization | 1.0 | 1.0 | 1.2 | 1.0 | 1.0 | 1.0 | 1.0 | 0.7 | 1.0 | 1.0 |
| | Initiator/ | | | | | | | | | | |
| | ((A) + Polymerization | | | | | | | | | | |
| | Initiator) | | | | | | | | | | |
| | (mass %) | | | | | | | | | | |

| | | | | | | | | | | |
|---|---|---|---|---|---|---|---|---|---|---|
| Total | 100.0 | 100.0 | 100.0 | 100.0 | 100.0 | 100.0 | 100.0 | 100.0 | 100.0 | 100.0 |
| [Surface hardness](HV0.2) | 34 | 32 | 30 | 41 | 45 | 32 | 30 | 31 | 30 | 41 |
| Compressive strength (Mpa) | 504 | 487 | 452 | 521 | 529 | 486 | 491 | 465 | 471 | 468 |
| Evaluation | A | B | B | A | A | B | B | B | B | B |
| Compressive displacement (mm) | 4.2 | 4.1 | 4.5 | 3.6 | 3.8 | 4.4 | 4.0 | 4.0 | 3.5 | 3.5 |
| Evaluation | A | A | A | B | B | A | A | A | B | B |
| Adhesive property to denture base material | B | A | A | A | A | A | A | A | B | A |

### Curable Compositions (Mass %) Used in Examples and Evaluation Results

TABLE 3

| | | Exam- | Exam- | Exam- | Exam- | Exam- | Exam- | Exam- | Exam- | Exam- | Exam- |
|---|---|---|---|---|---|---|---|---|---|---|---|
| | | ple 21 | ple 22 | ple 23 | ple 24 | ple 25 | ple 26 | ple 27 | ple 28 | ple 29 | ple 30 |
| Polymerizable | UDMA | — | — | 21.0 | 21.0 | 21.0 | 21.0 | 21.0 | 20.8 | 20.8 | 21.0 |
| monomer | UDA | 40.0 | — | — | — | — | — | — | — | — | — |
| (A) | Bis-GMA | — | 21.4 | — | — | — | — | — | — | — | — |
| | TEGDMA | 10.0 | 9.3 | 9.0 | 9.0 | 9.0 | 9.0 | 9.0 | 9.0 | 8.9 | 9.0 |
| | MMA | — | — | — | — | — | — | — | — | — | — |
| | Amount of | — | — | — | — | — | — | — | — | — | — |
| | MMA in (A) | | | | | | | | | | |
| Organic- | O1 | — | — | 45.0 | 45.0 | 45.0 | 45.0 | 45.0 | 45.0 | 45.0 | 45.0 |
| inorganic | O2 | — | 55.0 | — | — | — | — | — | — | — | — |
| composite | O3 | — | — | — | — | — | — | — | — | — | — |
| filler | O4 | — | — | — | — | — | — | — | — | — | — |
| (B) | O5 | — | — | — | — | — | — | — | — | — | — |
| | O6 | — | — | — | — | — | — | — | — | — | — |
| | O7 | — | — | — | — | — | — | — | — | — | — |
| | O8 | 25.0 | — | — | — | — | — | — | — | — | — |
| Inorganic fine | I1 | — | — | 3.7 | 3.7 | 3.7 | 3.7 | 3.7 | 3.7 | 3.7 | 3.7 |
| particle (C) | I2 | 21.5 | 9.0 | 18.0 | 18.0 | 18.0 | 18.0 | 18.0 | 18.0 | 18.0 | 18.0 |
| Other inorganic | I′1 | — | — | — | — | — | — | — | — | — | — |
| fine particle (C′) | I′2 | — | — | — | — | — | — | — | — | — | — |
| Non- | nCP1 | 3.0 | 5.0 | — | — | — | — | — | 3.0 | 3.0 | 1.5 |
| crosslinked | nCP2 | — | — | — | — | — | — | — | — | — | — |
| polymer | nCP3 | — | — | — | — | — | — | — | — | — | — |
| particle (D) | nCP4 | — | — | — | — | — | — | — | — | — | — |
| | nCP5 | — | — | — | — | — | — | — | — | — | — |
| | nCP6 | — | — | — | — | — | — | — | — | — | — |
| | nCP7 | — | — | 3.0 | — | — | — | — | — | — | — |
| | nCP8 | — | — | — | 3.0 | — | — | — | — | — | — |
| | nCP9 | — | — | — | — | 3.0 | — | — | — | — | — |
| | nCP10 | — | — | — | — | — | 3.0 | — | — | — | — |
| | nCP11 | — | — | — | — | — | — | 3.0 | — | — | — |
| Crosslinked | CP1 | — | — | — | — | — | — | — | — | — | 1.5 |
| polymer particle (D′) | CP2 | — | — | — | — | — | — | — | — | — | — |
| Polymerization | BPO | 0.5 | 0.3 | 0.3 | 0.3 | 0.3 | 0.3 | 0.3 | 0.5 | 0.6 | 0.3 |
| Initiator | Polymerization | 1.0 | 1.0 | 1.0 | 1.0 | 1.0 | 1.0 | 1.0 | 1.5 | 2.1 | 1.0 |
| | Initiator/ | | | | | | | | | | |
| | ((A) + Polymerization | | | | | | | | | | |
| | Initiator) | | | | | | | | | | |
| | (mass %) | | | | | | | | | | |

| | | | | | | | | | | |
|---|---|---|---|---|---|---|---|---|---|---|
| Total | 100.0 | 100.0 | 100.0 | 100.0 | 100.0 | 100.0 | 100.0 | 100.0 | 100.0 | 100.0 |
| [Surface hardness](HV0.2) | 38 | 37 | 31 | 31 | 32 | 33 | 34 | 36 | 37 | 30 |
| Compressive strength (Mpa) | 479 | 439 | 471 | 458 | 512 | 517 | 506 | 508 | 477 | 491 |
| Evaluation | B | B | B | B | A | A | A | A | B | B |
| Compressive displacement (mm) | 3.7 | 3.9 | 4.1 | 4.1 | 4.0 | 4.1 | 4.0 | 4.1 | 3.6 | 3.9 |
| Evaluation | B | B | A | A | A | A | A | A | B | B |
| Adhesive property to denture | A | A | A | A | A | A | B | A | A | A |
| base material | | | | | | | | | | |

### Curable Compositions (Mass %) Used in Comparative Examples and Evaluation Results

TABLE 4

| | | Compar- | Compar- | Compar- | Compar- | Compar- |
|---|---|---|---|---|---|---|
| | | ative | ative | ative | ative | ative |
| | | Exam- | Exam- | Exam- | Exam- | Exam- |
| | | ple 1 | ple 2 | ple 3 | ple 4 | ple 5 |
| Polymer- | UDMA | 23.1 | 21.0 | 21.0 | 21.0 | 19.6 |
| izable | UDA | — | — | — | — | — |
| monomer | Bis-GMA | — | — | — | — | — |
| (A) | TEGDMA | 9.9 | 9.0 | 9.0 | 7.5 | 4.2 |
| | MMA | — | — | — | 1.5 | 4.2 |
| | Amount of | — | — | — | 5.0 | 15.0 |
| | MMA in (A) | | | | | |
| Organic- | O1 | 45.0 | 45.0 | 45.0 | 46.0 | 40.0 |
| inorganic | O2 | — | — | — | — | — |
| composite | O3 | — | — | — | — | — |
| filler | O4 | — | — | — | — | — |
| (B) | O5 | — | — | — | — | — |
| | O6 | — | — | — | — | — |
| | O7 | — | — | — | — | — |
| | O8 | — | — | — | — | — |
| Inorganic fine | I1 | 3.7 | 3.7 | 3.7 | 4.2 | 4.0 |
| particle (C) | I2 | 18.0 | 18.0 | 18.0 | 19.0 | 20.7 |
| Other inorganic | I′1 | — | — | — | — | — |
| fine particle | I′2 | — | — | — | — | — |
| (C′) | | | | | | |
| Non- | nCP1 | — | — | — | 0.5 | 7.0 |
| crosslinked | nCP2 | — | — | — | — | — |
| polymer | nCP3 | — | — | — | — | — |
| particle (D) | nCP4 | — | — | — | — | — |
| | nCP5 | — | — | — | — | — |
| | nCP6 | — | — | — | — | — |
| | nCP7 | — | — | — | — | — |
| | nCP8 | — | — | — | — | — |
| | nCP9 | — | — | — | — | — |
| | nCP10 | — | — | — | — | — |
| | nCP11 | — | — | — | — | — |
| Crosslinked | CP1 | — | 3.0 | — | — | — |
| polymer | CP2 | — | — | 3.0 | — | — |
| particle | | | | | | |
| (D′) | | | | | | |
| Polymerization | BPO | 0.3 | 0.3 | 0.3 | 0.3 | 0.3 |
| Initiator | Polymerization | 0.9 | 1.0 | 1.0 | 1.0 | 1.1 |
| | Initiator/ | | | | | |
| | ((A) + | | | | | |
| | Polymerization | | | | | |
| | Initiator) | | | | | |
| | (mass %) | | | | | |
| Total | | 100.0 | 100.0 | 100.0 | 100.0 | 100.0 |
| [Surface hardness](HV0.2) | | 34 | 31 | 29 | 31 | 30 |
| Compressive strength (Mpa) | | 507 | 487 | 458 | 474 | 428 |
| Evaluation | | A | B | B | B | C |
| Compressive displacement (mm) | | 3.4 | 3.2 | 3.7 | 3.8 | 3.4 |
| Evaluation | | C | C | B | B | C |
| Adhesive property to denture base material | | C | C | C | C | A |
| | | Compar- | Compar- | Compar- | Compar- | Compar- |
| | | ative | ative | ative | ative | ative |
| | | Exam- | Exam- | Exam- | Exam- | Exam- |
| | | ple 6 | ple 7 | ple 8 | ple 9 | ple 10 |
| Polymer- | UDMA | 20.7 | 21.8 | — | — | — |
| izable | UDA | — | — | 14.7 | 27.1 | 18.4 |
| monomer | Bis-GMA | — | — | 8.8 | 16.3 | 11.0 |
| (A) | TEGDMA | 8.9 | 9.4 | 5.9 | 10.8 | 7.3 |
| | MMA | — | — | — | — | — |
| | Amount of | — | — | — | — | — |
| | MMA in (A) | | | | | |
| Organic- | O1 | 40.0 | 42.5 | 30.0 | — | 60.0 |
| inorganic | O2 | — | — | — | — | — |
| composite | O3 | — | — | — | — | — |
| filler | O4 | — | — | — | — | — |
| (B) | O5 | — | — | — | — | — |
| | O6 | — | — | — | — | — |
| | O7 | — | — | — | — | — |
| | O8 | — | — | — | — | — |
| Inorganic fine | I1 | 3.0 | — | — | 12.5 | — |
| particle (C) | I2 | 17.2 | 18.0 | — | 30.0 | — |
| Other inorganic | I′1 | — | 5.0 | — | — | — |
| fine particle | I′2 | — | — | 37.5 | — | — |
| (C′) | | | | | | |
| Non- | nCP1 | — | 3.0 | 3.0 | 3.0 | 3.0 |
| crosslinked | nCP2 | 10.0 | — | — | — | — |
| polymer | nCP3 | — | — | — | — | — |
| particle (D) | nCP4 | — | — | — | — | — |
| | nCP5 | — | — | — | — | — |
| | nCP6 | — | — | — | — | — |
| | nCP7 | — | — | — | — | — |
| | nCP8 | — | — | — | — | — |
| | nCP9 | — | — | — | — | — |
| | nCP10 | — | — | — | — | — |
| | nCP11 | — | — | — | — | — |
| Crosslinked | CP1 | — | — | — | — | — |
| polymer | CP2 | — | — | — | — | — |
| particle | | | | | | |
| (D′) | | | | | | |
| Polymerization | BPO | 0.2 | 0.3 | 0.1 | 0.3 | 0.3 |
| Initiator | Polymerization | 0.7 | 1.0 | 0.3 | 0.6 | 0.8 |
| | Initiator/ | | | | | |
| | ((A) + | | | | | |
| | Polymerization | | | | | |
| | Initiator) | | | | | |
| | (mass %) | | | | | |

| | | | | | |
|---|---|---|---|---|---|
| Total | 100.0 | 100.0 | 100.0 | 100.0 | 100.0 |
| [Surface hardness](HV0.2) | 28 | 37 | 45 | 38 | 27 |
| Compressive strength (Mpa) | 416 | 526 | 506 | 442 | 398 |
| Evaluation | C | A | A | B | C |
| Compressive displacement (mm) | 3.1 | 3.2 | 2.1 | 2.6 | 4.2 |
| Evaluation | C | C | C | C | A |
| Adhesive property to denture base material | A | B | B | A | A |

As shown in Tables 1 to 4, the curable compositions of the present disclosure in Examples 1 to 29 had high surface hardness and compressive strength, and had a large amount of compressive displacement and were less susceptible to fracture. The adhesive property to the denture base material was also excellent. On the other hand, the curable compositions of Comparative Examples 1 to 10 were inferior to the curable compositions of Examples 1 to 29 of the present disclosure in any one of the properties of the surface hardness, the compressive strength, the compressive displacement (fracture resistance) or adhesive property to the denture base material.

With respect to the use of substantially any plural and/or singular terms herein, those having skill in the art can translate from the plural to the singular and/or from the singular to the plural as is appropriate to the context.

Although the description herein has been given with reference to the drawings and embodiments, it should be noted that those skilled in the art may make various changes and modifications on the basis of this disclosure without difficulty. Accordingly, any such changes and modifications are intended to be included in the scope of the embodiments.

### INDUSTRIAL APPLICABILITY

The composite resin tooth of the present disclosure can be used for manufacturing dentures, such as complete dentures and partial dentures.

1. A composite resin tooth having a single layer structure or a layer structure of two or more layers, comprising
a composite resin layer consisting of a polymerized and cured product of a curable composition including polymerizable monomer (A), organic-inorganic composite filler (B), inorganic fine particle (C) and non-crosslinked polymer particle (D), wherein
the content of the non-crosslinked polymer particle (D) in the curable composition is within a range of 1% by mass or more and 5% by mass or less, and
an average particle diameter of all of the inorganic particles (C) contained in the curable composition is 1 μm or less.

2. The composite resin tooth according to claim 1, wherein
the average particle diameter of the non-crosslinked polymer particle (D) is 5 μm or more and 50 μm or less.

3. The composite resin tooth according to claim 1, wherein
the content of the inorganic fine particles (C) in the curable composition is within a range of 15% by mass or more and 35% by mass or less, and
the content of the inorganic filler (b-1) contained in the organic-inorganic composite filler (B) is within a range of 10% by mass or more and 35% by mass or less.

4. The composite resin tooth according to claim 1, wherein,
the content of methyl methacrylate with respect to the whole of the polymerizable monomer (A) is 5 mass % or less.

5. The composite resin tooth according to claim 3, wherein,
the content of methyl methacrylate with respect to the whole of the polymerizable monomer (A) is 5 mass % or less.

6. The composite resin tooth according to claim 1, wherein,
the non-crosslinked polymer particle (D) includes a polymethyl methacrylate particle.

7. The composite resin tooth according to claim 3, wherein,
the non-crosslinked polymer particle (D) includes a polymethyl methacrylate particle.

8. The composite resin tooth according to claim 4, wherein,
the non-crosslinked polymer particle (D) includes a polymethyl methacrylate particle.
